(19) United States
(12) Patent Application Publication
(10) Pub. No.: US 2002/0048553 A1
(43) Pub. Date: Apr. 25, 2002

(54) Toothpaste

(76) Inventor: Irene Baumgartner, Schwabach (DE)

Correspondence Address: Patent Attorney's Gotz & Kuchler, Farberstrabe 20, Nurnberg, 90402, DE

(21) Appl. No.: 09/917,513

(22) Filed: Jul. 30, 2001

(30) Foreign Application Priority Data
Aug. 17, 2000 (DE) 100 40 133.3

(51) Int. Cl. A61K 7/16

(52) U.S. Cl. 424/49

(57) ABSTRACT

The herein-described invention promotes oral and dental health by encouraging children to brush longer, therewith promoting healthier gums and preventing tooth decay. Therefor, the invention provides a toothpaste containing capsules or particles filled with child-friendly flavorings and dyes which will burst open or liquefy through the motion and pressure of brushing to release their tasty contents as a reward for an intense brushing.

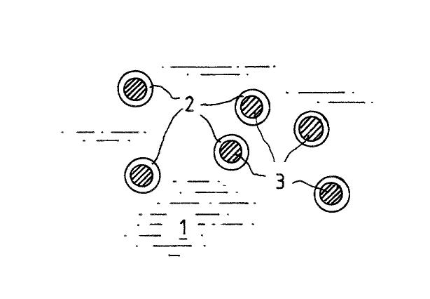

### BACKGROUND OF THE INVENTION

1. Field of the Invention

The invention refers to a toothpaste, especially for children, that contains active ingredients as well as flavors and eventually dyes.

2. Description of the Related Art

Regular brushing is the key to a good dental hygiene. That is not only true for the teeth of adults, but also for the primary dentition of children. Without regular brushing, cavities are predictable. Motivating children to brush can be a difficult task; yet it is an important one. To encourage children in their brushing, flavors have been added to a toothpaste. However, this is not enough, because children have the tendency to swallow the flavored toothpaste instead of brushing, sometimes they even choose to swallow consciously. Parents can attest to this. If swallowing the toothpaste, children are not receiving the necessary fluorine to prevent tooth decay. To harden enamel, fluorine is added to children's toothpaste. The amount of fluorine added is within prescribed limits to prevent an unhealthy overdose of fluorine ingestion. This is important as the majority of children under the age of two receive additional doses of fluorine through a tablet form, fluorine enhanced table salt or fluoridated water. Through these means, the amount of fluorine a child receives is within the acceptable limits. Too much fluorine can be as dangerous as not enough. Ingesting an unnecessary amount of fluorine that their developing bodies not need or benefit from can lead to other possible medical concerns. Consequently, it becomes necessary to police the amount of fluorine ingested, to make sure the child does not “snack” on the sweet fruity and enticing taste. To date, only two unacceptable facts exist, the first, that the colorful, sweet toothpaste is eaten and swallowed more than used for thorough brushing, and second, that an overingestion of fluorine can result in a variety of harmful effects.

Drawn from the disadvantages of current practice, the problem arises to improve a known toothpaste so that by the tasty ingredients children will be motivated to a thorough brushing for removing plaque, instead of swallowing the toothpaste.

### BRIEF SUMMARY OF THE INVENTION

This problem is solved by creating a type of toothpaste, whereby the tasty ingredients and eventually the dye-stuff or pigment ingredients are solved or diluted by an alcohol-free solution agent and are enclosed within capsules or bound in particles of binding agent, which capsules and/or binding particles are spread uniformly within the creamy toothpaste mass and will break open under mechanical influence, especially by the motion and pressure applied through brushing, to set their tasty and/or colored ingredients free.

The purpose for encapsulating the tasty and eventually colored ingredients in capsules and/or binding particles is that these capsules and/or particles have to be broken up for enjoying the pleasant taste. Therefore, the child has to brush in order to enjoy the flavor and/or color. The capsules and/or binding particles are constructed in such a way that they will only burst open through applied motion and/or pressure, which is a part of the motion and pressure of tooth brushing. The tasty ingredients can only be sensed after serious tooth brushing and not already with partaking of the toothpaste. Thereby the solution agent provides for a fast distribution of the tasty ingredients. The active ingredients, i.e. fluorine, are outside of the capsules or particles for immediate application. The children are “rewarded” for a serious brushing. Each child will brush more just to sense the formerly tasteless toothpaste converting into a tasty mass, and/or to see his or her mouth covered in colored foam in the mirror.

The construction of the capsules is rather variable: As long as a breaking open under mechanical influence is possible, the hardness of the capsules may vary within a limited range. If the capsule casing is able to withstand the gel or cream toothpaste in which it is contained, any substance may be used which is naturally safe and sound. The content of the capsules may have a different color than the gel or cream paste to allow third persons to determine whether a proper brushing has been done. So a mother may check whether her child has brushed with the necessary pressure.

For optimal application, the diameter of the capsules or particles should measure between 0,1 mm and 4 mm, preferably between 0,5 mm and 3 mm or optimally between 1 mm and 2 mm. Capsules/particles in this preferred size range are easily recognizable as well as easily sensed making it easy for the child to break open the capsules through the motion of brushing. The size of the child's tooth combined with the brush will allow for optimal release or bursting of the particles, and for easy dispensing from the toothpaste tube.

The ratio of the particle volume to toothpaste volume should be between 10%-50%, preferably between 20%-45%, and optimally between 30%-40%. The ratio should not be too high; it should not impede the release of the paste from the tube.

Nor should the ratio be too low; it should cause a child to brush over a time of about 5 minutes to achieve the goals of sound dental hygiene, prevention of tooth decay and cavities, through active brushing.

The capsules and/or particles could be made of a thixotropic gel and possibly of an active ingredient. The flavor particles should be stable enough to withstand the production process, storage and removal from the tube. It might be more beneficial to use a cylinder that pushes up from the bottom rather than a tube requiring a is squeeze to retrieve the gel or cream toothpaste from the tube without rising the pressure in any region of the toothpaste over a level which could cause breaking the capsules or particles. Therefore, if pressure is applied, the capsules must be able to pass this pressure on to their contents. A gelatine capsule might work well for this purpose owing to their deformability. On the other side, bursting or breaking of the particles through brushing could be promoted, as a substance with a thixotropic property liquidifies because of deformations and releases its tasty content.

A capsule is preferably composed of a jelly-like material, especially of an organic colloid, preferably originating from an animal source, such as gelatine, or from a plant substance, such as agar-agar, pectin, etc. These materials are safe enough in the event that some toothpaste is swallowed. Generally a small amount of perhaps 0,5 vol.-% to 1 vol.-% of a gelatinous substance is sufficient, especially if it is mixed into warm or hot water to form a jelly-like substance after the cooling phase. These types of capsules could be manufactured in a way that the flavoring and dye is injected through a specially designed nozzle into the jelly-like substance simultaneously as the jelly-like substance cures, so that the drops of flavoring will be covered by the jelly-like substance.

For the purpose of storage stability, the substance of the capsules/particles should not be water soluble at room temperature. This will allow the capsules to be combined with water-based toothpaste, which is both healthy and cost-efficient. As gelatine dissolves at higher temperatures, it could be designed in such a way to dissolve at the regular temperature of the human body. Generally tooth enamel does not reflect the body temperature of 37° C., and this temperature is already lower than an extended summertime heat-wave which exceeds 37° C. Consequently the optimal temperature for water dissolution should be above 35° C.-40° C. The capsules should not break open when the toothpaste comes in contact with the gum, but through the motion and applied pressure of brushing.

It is proven successful that natural flavoring, such as essential oils, but also artificial flavoring can be used. Using flavors and aromas that children are familiar with, i.e. strawberry, will make brushing more appealing, as the dispensation of strawberry aroma leads children to think of the joyful consumption of strawberries. For small children, an appealing but unfamiliar taste could encourage brushing and prevent uncritical swallowing.

A paste sweetened with a sugar substitute also is beneficial in a way that no additional calories would be ingested and of course, would not promote caries, while the sweet taste improves the attraction of such toothpaste for children.

To increase the durability of the toothpaste according to the invention, a flavor and/or sweetener conserving agent could be added. The conserving agent used should prevent oxidation of natural flavors and/or sweeteners.

The ratio of essential oil to 100 ml of toothpaste should be between 0,05 ml to 1 ml, preferably 0,1 ml to 0,5 ml, optimally 0,2 ml to 0,4 ml. The remainder of the cavities within the capsules would be filled with the solvent. Essential oils are a good choice as they can imitate almost any available natural flavor. On the other side, essential oils have an intensive taste and should only be used in small amounts. Therefore, a solution of such essential oils in an aqueous solvent is absolutely necessary.

The coloring could be found within the capsules, in the wall of the capsules, or in the particles made of binding agent, but preferably in the wall of the capsules. The coloring can serve to make the flavor capsules/particles optically distinguishable from the creamy toothpaste mass in order that a child can aim the brushing activity to the uncrushed particles, by watching himself/herself in the mirror. On the other side, this or another dye could be mixed with the flavoring in the capsules itself so that it will be dispensed together with the aroma only during intense brushing. This will help the caregiver to determine if enough brushing has already taken place.

Complete prevention of the ingestion of some toothpaste by some adventuresome children is not possible. So to comply with standard health practices, only ingestible food dye should be used.

The volume of dye used influences the intensity of the color. Good results can be expected when the ratio of dye to toothpaste is about 1 ml to 15 ml per 100 ml of toothpaste, preferably 2 ml to 10 ml/100 ml of paste, optimally 4 ml-6 ml. The level of color released through the flavor particles determines the amount of dye. Pink and light green are good choices, which allow for maximum success with a very small amount of dye. Darker colors, such as red or blue, require less dye than for example yellow.

The design of the invention allows for flavor and color to be adjusted independently from each other. Taste could be optimized while the coloring complements it. The coloration of the toothpaste by breaking the capsules/particles releasing the taste and dye particles could awaken a recognition effect in the child. If color and taste complement one another, the child could recognize a preferred fruit flavor. For this purpose, the following aromas and colors could be combined: The taste of strawberries, with light red; cherry flavor could be combined with dark red; orange flavored paste could be combined with orange color, etc. The combination of flavor and color intensity could be adjusted accordingly to the intended extent.

To prevent the capsules from bursting prematurely, a thickening agent can be used. Thereby, the viscosity of liquid substances like essential oils solved in water can be increased, for example like the consistency of honey. Incorporation of a tickening increased, for example like the consistency of honey. Incorporation of a thickening agent in the water-soluble mass would allow for the capsules/particles to maintain their spherical, drop-like shape, until the desired mechanical application through the pressure and motion of brushing occurs. Such pressure and motion of brushing will cause a deformation of the capsules/particles until their elastic deformability is exceeded, for example, through the thixotropic characteristics triggered by the pressure of brushing, and the capsules break open.

A water-soluble thickening agent promotes the dissolution of the content of a burst capsule or particle mass within the mouth cavity. As the toothpaste composition can mostly be water, enough solvent is present to convert the capsule mass into a liquid state releasing the color flavored particles within the mouth to reward the child for appropriate brushing practice.

So that the flavor particles could be easily recognized, the toothpaste itself could be transparent or white. A transparent paste would allow for easy verification that the capsules/particles containing the flavor/color are still intact during production and storage stages.

So that they can be most effective, the fluoride and other active ingredients should be exterior of the capsules/particles in the cream-like toothpaste component. Application of the fluoride and other active ingredients is then not dependent on the brusher's ability to break and release the capsules/particles through the pressure and motion of brushing.

The concentration of the active ingredients can be increased in correspondence to the portion of flavor/color capsules and/or particles of binding agent so that the volume specific health-care effect of the toothpaste will not be decreased. Therefore, the concentration of active ingredients in the mixture of toothpaste mass and released capsule contents is equal to the concentration of usual toothpastes and will not require an increase of brushing time.

The toothpaste may contain abrasive particles, which aid in the removal of plaque and could help to break open the capsules/particles. Such abrasive particles could be made of silicon oxide, for example, which is not poisonous and maintains hardness at room temperature. To easily differentiate the abrasive particles from the flavor/color beads, the abrasive particles should measure smaller than the flavor capsules/particles, preferably 1-50 μm, optimally 2-20 μm. The smaller size will also assist in an optimal crushing of the flavor/color capsules/particles.

To avoid an unhealthy ingestion of fluorine through an over use of toothpaste, the volume of fluoride, for example, sodium fluoride or sodium mono fluoride phosphate, should be less than 1 weight-%, preferably less than 0,3 weight-% and optimally less than 0,1 weight-% of the toothpaste. As fluoride can have harmful side effects, and children are likely to swallow toothpaste, a reduction in the volume of fluoride can help to reduce the possibilities that children suffer harmful effects. The fluoride ratio to paste in children and adolescent toothpaste should be less than 1 mg per gram of toothpaste.

Finally, the alcohol content should be smaller than 2%, preferably smaller than 1%, optimally, less than 0,5% of the total tooth paste mass. The use of (ethyl) alcohol should be avoided in the toothpaste base as much as possible, in order to prevent an alcohol addiction through the regular swallowing of toothpaste.

### BRIEF DESCRIPTION OF THE DRAWINGS

Additional features, details, benefits and effects on the basis of this invention are further discussed in the additional description of a preferred example of the invention with regard to the accompanied drawing which shows a part of a toothpaste according to the invention.

### DETAILED DESCRIPTION OF THE INVENTION

The toothpaste according to the invention herein described is composed of three components: The cream-like base component 1 of the toothpaste; the capsules 2 which are distributed within the cream-like base component 1, and the tasty contents 3 of the capsules 2.

In the following, a preferred composition of these components 1-3 is listed in weight-% unless otherwise stated.

1. Toothpaste Composition (50-80 vol.-% of the Toothpaste)

| | |
|---|---|
| Moist-keeping and/or thickening agent, natural or synthetic | 10-40% |
| gum such as Irish moss, sodium carboxymethyl cellulose, | |
| methylcellulose or hydroxyethylcellulose, as well as guar | |
| gum, polyvinylpyrollidon and starch | |
| Active ingredients against dental plaque and bacteria, etc. | 0.2-10% |
| Sodium mono fluorine phosphate | 1-2 ppm |
| Abrasive agent (i.e. colloid silicon oxide) | 30-50% |
| Disinfection agent | about 1.5% |
| Sweetener, i.e. saccharin | about 0.03% |
| Whitener | as needed |
| Preservative | as needed |
| Agent to establish PH balance, i.e. NaOH | as needed |
| H₂O (Water) | balance |

The proportions listed are based on 100% of the cream-like toothpaste component.

2. Capsule/particle Covering (5-20 vol.-% of the Toothpaste)

| | |
|---|---|
| Gelatinous substance, such as, gelatine, agar-agar, etc. | 0.5-2% |
| Softener (possibly thixotropic like plant oils (cocoa or | as needed |
| peanut oil), beeswax, etc.) | |
| H₂O (water bound in gelatinous substance) | balance |

These proportions are weight based and based on 100% of the capsule covering.

3. Flavor Substance in the Capsules/particles (20-50 vol.-% of the Toothpaste)

| | |
|---|---|
| Essential oils, i.e. from sage, peppermint, eucalyptus, fennel, | 2-10% |
| strawberry, orange, lemon or camomille | |
| Food dye, powdered form | 5-15% |
| Thickening agent (could be thixotropic) | 0-30% |
| Preservative | as needed |
| H₂O (Water) | balance |

These proportions are based on weight and on 100% of the capsule filling component.

1. Toothpaste with dental health promoting active ingredients as well as flavor and/or dyes, characterized in that the flavor ingredients are liquefied by and/or solved in an alcohol-free solvent, and are encapsulated in solid or gelatine-like capsules and/or bound in particles consisting of binding agent; whereby the capsules or particles are evenly distributed through the cream-like toothpaste base and can be broken open or liquefied through the pressure and motion of brushing.

2. Toothpaste according to claim 1, characterized in that the diameter of the capsules or particles measures between 0,1 mm and 4 mm, preferably between 0,5 mm and 3 mm, and optimally between 1 mm and 2 mm.

3. Toothpaste according to claim 1, characterized in that the number of capsules or particles is designed so that their total volume does not exceed 10-50% of, preferably 20-45% of, optimally 30-40% of the total toothpaste volume.

4. Toothpaste according to claim 1, characterized in that the capsules or particles are made from a thixotropic material.

5. Toothpaste according to claim 1, characterized in that the capsules have a gelatine-based shell, especially made of organic colloids, preferably of animal origin like gelatine, or plant-like origin, such as agar-agar, pectin, etc.

6. Toothpaste according to claim 5, characterized in that the substance of the capsule shell is not water soluble at room temperature.

7. Toothpaste according to claim 1, characterized by the use of natural aromas like essential oils, or of natural-like flavorings which are identical with natural flavorings, and/or of artificial flavors.

8. Toothpaste according to claim 7, characterized in that the volume of an essential oil per 100 ml toothpaste is approximately equal to 0,05 ml-1 ml, preferably 0,1 ml to 0,5 ml, optimally 0,2 ml to 0,4 ml.

9. Toothpaste according to claim 1, characterized in that the flavor component comprises a sugar substitute.

10. Toothpaste according to claim 1, characterized in that the capsules, specifically the solvent, contain a flavor and/or sweetener chemically stabilizing preservative.

11. Toothpaste according to claim 1, characterized in that the dye-stuff is contained within the capsules, in the wall of the capsules, or in the particles consisting of binding agent.

12. Toothpaste according to claim 11, characterized in that the coloring agent is food dye.

13. Toothpaste according to claim 11, characterized in that the volume of dye-stuff per 100 ml toothpaste is approximately equal to 1 ml-15 ml, preferably 2 ml-1 0 ml, preferably 4 ml-6 ml.

14. Toothpaste according to claim 1, characterized in that flavor and food coloring are adjustable independently of one another to optimize the flavor, and also to adapt the color to the taste used to flavor the toothpaste.

15. Toothpaste according to claim 1, characterized in that the capsule contents is stabilized by a thickening agent.

16. Toothpaste according to claim 1, characterized in that the toothpaste base is white or transparent.

17. Toothpaste according to claim 1, characterized in that dental health care active ingredients are primarily or entirely in the cream-like toothpaste base outside of the capsules and/or particles.

18. Toothpaste according to claim 17, characterized in that the concentration of the active ingredients in the toothpaste base mass is increased corresponding to the volume of the capsules and/or particles of binding agent.

19. Toothpaste according to claim 1, characterized in that abrasive particles are contained in the toothpaste base mass, which serve to remove plaque and assist in the breakdown of the capsules or particles.

20. Toothpaste according to claim 1, characterized in that the volume of fluorine containing components, i.e. sodium fluoride or sodium mono fluoride phosphates limited to less than 1% of the volume, preferably less than 0,3%, and optimally less than 0,1% of the total volume.
